(19) United States
(12) Patent Application Publication
(10) Pub. No.: US 2022/0065848 A1
(43) Pub. Date: Mar. 3, 2022

(54) SUBSTRATE FOR AMPLIFYING THE CHEMILUMINESCENCE

(71) Applicant: Fianostics GmbH, Wiener Neustadt (AT)

(72) Inventors: Albert Missbichler, Vienna (AT); Gerhard Hawa, Vienna (AT)

(73) Assignee: Fianostics GmbH, Wiener Neustadt (AT)

(21) Appl. No.: 17/421,119

(22) PCT Filed: Mar. 18, 2020
(86) PCT No.: PCT/AT2020/060112

§ 371 Date: Jul. 7, 2021

(30) Foreign Application Priority Data
Apr. 3, 2019 (AT) A 50290/2019

(51) Int. Cl. G01N 33/533; G01N 33/58; G01N 33/553; G01N 33/545; C12N 9/08; C12N 9/02; C12N 9/16

(52) U.S. Cl. CPC G01N 33/533; G01N 33/581; G01N 33/582; G01N 33/553; G01N 21/76; C12N 9/0065; C12N 9/0069; C12N 9/16; G01N 33/545

(57) ABSTRACT

The present invention relates to the use of a substrate for enhancing the chemiluminescence of one or more luminophores produced in a chemiluminescence reaction, wherein the substrate comprises a solid polymeric carrier with a plurality of depressions separated from one another and that the solid carrier is at least partially coated with a metal.

The present invention relates to the use of a substrate with a nanostructured surface for enhancing chemiluminescence. This effect is also known as “metal-enhanced chemiluminescence” (MEC).

Chemiluminescence is a process in which electromagnetic radiation in the ultraviolet and/or visible light range is emitted through a chemical reaction. Usually, oxidation-sensible compounds are involved in chemiluminescence reactions, which first oxidize to become instable intermediate compounds, in order to release light in a subsequent reaction. Such compounds are also referred to as luminophores. A well-known and frequently used luminophore is luminol. It is, inter alia, used in forensics because luminol allows the detection of blood residues. This is based on the fact that after oxidation with an oxidant, luminol reacts by emitting bluish light. However, only in the presence of catalysts (e.g. complex-bound Fe³⁺) this reaction takes place at a sufficient rate, i.e. in the absence of catalysts, no reaction and thus no chemiluminescence is observable. Luminophores may also be produced through the reaction of certain compounds in the presence of enzymes. The fact that enzymes are able to produce luminophores is e.g. used in analytics. In ELISA (enzyme-linked immunosorbent assay) applications, for example, molecules binding analytes several times are coupled with corresponding enzymes in order to determine the presence of certain analytes in a sample by producing luminophores.

In particular in analytic procedures, it is of great importance to determine even small amounts of an analyte in a sample. Conventional methods for determining and quantifying analytes in a sample often have the disadvantage that they are not able to detect analyte concentrations in the nanogram or picogram range.

It is therefore an object of the present invention to provide means and methods that are able to increase the sensitivity of the previously described analytical methods.

This object is achieved by using a substrate for enhancing the chemiluminescence of one or more luminophores produced in a chemiluminescence reaction, the substrate comprising a solid polymeric carrier with a plurality of depressions separated from one another and the solid carrier being at least partially coated with a metal.

Such substrates are known for enhancing fluorescence, e.g. from WO 2017/046320. With the nanostructured surfaces of these substrates it is possible to significantly increase the fluorescence yield of fluorescent compounds, which drastically increases the sensitivity of fluorescence measurements through a so-called MEF (“metal-enhanced fluorescence”) effect. This allows measuring very low fluorophore concentrations. However, this fluorescence enhancement is only observable when the fluorophore is in close proximity (less than 50 nm) to the substrate (Hawa et al. Analytical Biochemistry 549(2018): 39-44). This MEF effect is only observable when fluorescence is measured through the transparent substrate or directly at the surface after removal of any solution present that is not surface-bound and thus contains non-enhanced fluorophore. Enhancement through an MEF effect of molecules that are free in solution is thus not possible.

However, it has surprisingly been shown that surface structures that are able to enhance the fluorescence of fluorescent materials may also be used to enhance electromagnetic emission of luminophores produced in a chemiluminescence reaction. However, it has been shown that the luminescence enhancing effect is not only observable when the luminophore is in close proximity (i.e. less than 50 nm) to the substrate described above and that the effect extends to the entire solution. It can thus also be observed when measuring from above through the entire bulk solution. Contrary to analytic methods where fluorescence of a fluorophore indirectly bound to a solid carrier is determined (e.g. ELISA), luminophores that are produced by enzymes indirectly bound to a solid carrier are free in solution and may diffuse freely in the aqueous solution to be examined. As a result, most luminophores in a reaction set-up are not in close proximity to the substrate but diffuse away from it. Despite this diffusion effect, it has been shown that the inventive substrate unexpectedly significantly enhances the chemiluminescence of the luminophores. In view of the findings of Hawa et al. (Analytical Biochemistry 549(2018): 39-44), this was not to be expected because the authors of that publication were able to show that a fluorescence-enhancing effect is only observable in close proximity to the inventive substrate.

The inventive solid carriers including depressions may basically be produced with different methods, as is also presented in WO 2017/046320.
(a) The solid carriers including the depressions are manufactured in one single step (e.g. injection molding)
(b) The depressions are introduced into an existing solid carrier in further process steps (e.g. hot embossing, electron beam lithography or “extreme ultraviolet” (EUV) in connection with reactive ion etching or laser ablation)
(c) To a solid carrier, a thin structurable polymer layer is applied, into which the depressions are introduced, such as during production of BD-50 Blu-ray Discs (UV nanoimprint lithography)

So-called nanoimprint lithography is particularly well suited for producing these structures (Chou S. et al., Nanoimprint lithography, Journal of Vacuum Science & Technology B Volume 14, Nr. 6, 1996, S. 4129-4133). For producing nanostructures by means of nanoimprint lithography, a positive, usually a monomer or polymer, and a nanostructured stamp (“master”) are required. The stamp itself may be produced by means of nanolithography, or alternatively it may be produced by etching. The positive is applied to a substrate and then heated to above the glass temperature, i.e. it is liquefied, before the stamp is impressed. In order to achieve controllable (and short-term) heating, laser or UV light are often used. Due to the viscosity of the positive during heating, the interspaces of the stamp are completely filled therewith. After cooling, the stamp is removed. The positive, which constitutes the solid carrier of the inventive substrate, is coated with metal by means of a sputtering method.

Structuring of the stamp for lithography may again be achieved by nanoimprinting. Here, the materials used are glass or light-transparent plastic.

Particularly preferred is the production of the solid carrier including depressions by means of injection molding. Here, the mold inserts are typically drawn from a lithographically produced Si wafer by means of Ni electroplating.

The solid carrier may basically have any shape (e.g. spherical, planar), with a planar shape being particularly preferred.

A “depression,” as used herein, relates to the level of the surface of the solid carrier surrounding the depression and extends into to carrier, not from it like an elevation or bump. A depression in the sense of the present invention has a bottom limited by side walls. Its depth is thus the distance from the surface to a bottom of the depression. The depressions on the solid carrier may have different shapes (e.g. round, oval, square, rectangular).

A “plurality” of depressions, as used herein, means that the inventive carrier has at least one, preferably at least two, more preferably at least 5, more preferably at least 10, more preferably at least 20, more preferably at least 30, more preferably at least 50, more preferably at least 100, more preferably at least 150, more preferably at least 200 depressions. These depressions may be provided on a surface area of the solid carrier of 1000 μm², preferably of 500 μm², more preferably of 200 μm², more preferably of von 100 μm². Alternatively, the depressions may extend over a length of preferably 1000 μm, more preferably of 500 μm, more preferably of 200 μm, more preferably of 100 μm.

“Depressions separated from one another,” as used herein, means that the depressions are separated from one another by their side limits and are not connected to each other—not even at the surface of the solid carrier.

According to a preferred embodiment of the present invention, the depressions have a distance (“period”) from each other of 0.2 μm to 2.5 μm, preferably of 0.3 μm to 1.4 μm, more preferably of 0.4 μm to 1.3 μm, auf. In a further preferred embodiment of the present invention, the depressions have a distance from each other of 0.2 μm to 2 μm, preferably of 0.2 μm to 1.8 μm, preferably of 0.2 μm to 1.6 μm, preferably of 0.2 μm to 1.5 μm, preferably of 0.2 μm to 1.4 μm, preferably of 0.2 μm to 1.3 μm, preferably of 0.3 μm to 2,5 μm, preferably of 0.3 μm to 2 μm, preferably of 0.3 μm to 1.8 μm, preferably of 0.3 μm to 1.6 μm, preferably of 0.3 μm to 1.5 μm, preferably of 0.3 μm to 1.3 μm, preferably of 0.4 μm to 2,5 μm, preferably of 0.4 μm to 2 μm, preferably of 0.4 μm to 1.8 μm, preferably of 0.4 μm to 1.6 μm, preferably of 0.4 μm to 1.5 μm, preferably of 0.4 μm to 1.4 μm, preferably of 0.5 μm to 2.5 μm, preferably of 0.5 μm to 2 μm, preferably of 0.5 μm to 1.8 μm, preferably of 0.5 um to 1.6 μm, preferably of 0.5 μm to 1.5 μm, preferably of 0.5 μm to 1.4 μm, preferably of 0.5 μm to 1.3 μm, preferably of 0.6 μm to 2,5 μm, preferably of 0.6 μm to 2 μm, preferably of 0.6 μm to 1.8 μm, preferably of 0.6 μm to 1.6 μm, preferably of 0.6 μm to 1.5 μm, preferably of 0.6 μm to 1.4 μm, preferably of 0.6 μm to 1.3 μm, preferably of 0.7 μm to 2,5 μm, preferably of 0.7 μm to 2 μm, preferably of 0.5 μm to 1.8 μm, preferably of 0.7 μm to 1.6 μm, preferably of 0.7 μm to 1.5 μm, preferably of 0.7 μm to 1.4 μm, preferably of 0.7 μm to 1.3 μm, wherein the depressions most preferably have a distance from each other of 0.2 μm to 1.4 μm or 0.3 μm to 1.3 μm. The distance between the depressions (“period”) is measured from the center of the depression.

According to a preferred embodiment of the present invention the depressions of the solid carrier have a length and a width, wherein the length-to-width ratio is 2:1 to 1:2, in particular approx. 1:1.

Essentially, the depressions on the solid carrier may have any shape. However, particularly preferred are depressions having a length-to-width ratio of 2:1 to 1:2, preferably 1,8:1, preferably 1,6:1, preferably 1,5:1, preferably 1,4:1, preferably 1,3:1, preferably 1,2:1, preferably 1,1:1, preferably 1:1,8, preferably 1:1,6, preferably 1:1,5, preferably 1:1,4, preferably 1:1,3, preferably 1:1,2, preferably 1:1,1, in particular 1:1.

According to a further preferred embodiment of the present invention, the length and width of the depressions are 0.1 μm to 2 μm, preferably 0.2 um to 2 μm, preferably 0.3 μm to 2 μm, preferably 0.1 μm to 1.8 μm, preferably 0.2 μm to 1.8 μm, preferably 0.3 μm to 1.8 μm, preferably 0.1 μm to 1.5 μm, preferably 0.2 μm to 1.5 μm, preferably 0.3 μm to 1.5 μm, preferably 0.1 μm to 1.2 μm, preferably 0.2 μm to 1.2 μm, preferably 0.2 μm to 1.2 μm, preferably 0.1 μm to 1 μm, preferably 0.2 μm to 1 μm, preferably 0.3 μm to 1 μm, preferably 0.1 μm to 0.8 μm, preferably 0.2 μm to 0.8 μm, preferably 0.3 μm to 0.8 μm, preferably 0.1 μm to 0.6 μm, preferably 0.2 μm to 0.6 μm, preferably 0.3 μm to 0.6 μm, in particular 0.2 μm to 0.6 μm.

In particular, the depressions of the inventive solid carrier have an essentially round shape, wherein “essentially round” also includes oval and ellipsoid shapes. The shape of the depression is visibly in a plan view of the solid carrier.

The depressions preferably have a depth of 0.1 μm to 5 μm, preferably of 0.1 μm to 4 μm, preferably of 0.1μm to 3 um, preferably of 0.1 μm to 2 μm, preferably of 0.1 μm to 1.5 μm, preferably of 0.1 μm to 1.2 μm, preferably of 0.1 μm to 1 μm, preferably of 0.1 μm to 0.9 μm, preferably of 0.1 um to 0.8 μm, preferably of 0.2 μm to 5 μm, preferably of 0.2 μm to 4 μm, preferably of 0.2 μm to 3 μm, preferably of 0.2 μm to 2 μm, preferably of 0.2 μm to 1.5 μm, preferably of 0.2 μm to 1.2 μm, preferably of 0.2 μm to 1 μm, preferably of 0.2 μm to 0.9 μm, preferably of 0.2 μm to 0.8 μm, preferably of 0.3 μm to 5 μm, preferably of 0.3 μm to 4 μm, preferably of 0.3 μm to 3 μm, preferably of 0.3 μm to 2 μm, preferably of 0.3 μm to 1.5 μm, preferably of 0.3 μm to 1.2 μm, preferably of 0.3 μm to 1 μm, preferably of 0.3 μm to 0.9 μm, preferably of 0.3 μm to 0.8 μm, auf. The depth of the depression is the distance of the surface of the solid metalized carrier to the bottom of the depression.

According to the invention, the solid polymeric carrier is “at least partially” covered with a metal. “At least partially,” as used herein, means at least 20%, preferably at least 30%, more preferably at least 40%, more preferably at least 50%, more preferably at least 60%, more preferably at least 70%, more preferably at least 80%, more preferably at least 90%, more preferably at least 95%, more preferably at least 98%, most preferably 100% of any area of the solid carrier comprising depressions is covered with at least one metal. Since the MEF effect requires a metallic surface, it is particularly preferred that the surface of the solid carrier is covered with at least one metal at least in the area of the depressions. Here, the solid carrier may also comprise several (e.g. at least two, at least three, at least four or at least five) metal layers arranged above one another made of the same or different metals. An advantage of using several metal layers on the solid carrier is that the first metal layer (e.g. chrome), which is applied directly to the carrier, can improve adherence of the further metal layers.

The term “arranged one above another,” as used herein, means that a metal layer is directly or indirectly arranged on another metal layer. This leads to a multilayer system of metal layers of the same metal or of different metals.

The metal layers are preferably continuous and not discontinuous. According to the invention it was, however, found that the metal layer or metal layers on the solid polymeric carrier may be discontinuous without compromising the fluorescence-enhancing effect. The discontinuous metal layer may, for example, result from a conductivity measurement of the surface of the inventive substrate. Lower or no conductivity means that the metal layer(s) is/are discontinuous at the substrate surface. Discontinuous metal layers may, for example, be produced by contacting a substrate that is essentially fully covered with metal with a preferably saline solution, such as 10 mM phosphate buffer with 150 mM NaCl, for a certain period of time (10-90 minutes).

The solid carrier of the present invention is “coated with at least one metal.” Preferably, the metal layer comprises at least two, more preferably at least three, more preferably at least four, more preferably at least five different metals. The metals may be applied to the solid carrier by means of methods known from the state of the art, wherein sputtering (cathode sputtering) or thermal evaporation, electron beam evaporation, laser beam evaporation, arc evaporation, molecular beam epitaxy, ion beam assisted deposition and ion plating are preferably used.

According to a preferred embodiment of the present invention, the metal is selected from the group consisting of silver, gold, aluminum, chrome, indium, copper, nickel, palladium, platinum, zinc, tin, and alloys comprising one or more of these metals.

According to the invention, these metals or alloys thereof may be used for coating the inventive solid carrier. Particularly preferred are coatings of the solid carrier with silver or alloys containing silver because silver and its alloys have a particularly strong enhancement effect. Particularly preferred is an alloy comprising silver, indium and tin. Silver-containing alloys preferably have a silver content of more than 10%, more preferably of more than 30%, more preferably of more than 50%, more preferably of more than 70%, more preferably of more than 80%, more preferably of more than 90%.

After coating the solid carrier with at least one metal or before using the inventive substrate or the inventive solid carrier, the solid carrier or the substrate is preferably treated with an aqueous composition comprising at least one acid or one salt of a halogen selected from the group consisting of fluorine, chlorine, bromine and iodine.

It has been shown that fluorescence enhancement may be enhanced even more by pre-treating the substrate or the solid carrier with an aqueous solution (e.g. a buffer) comprising at least one acid of a halogen or a salt thereof. Therefore, it is particularly preferred to pre-treat the solid carrier or the substrate with an acid- or salt-containing solution. Alternatively, the aqueous solution (e.g. a buffer) comprising at least one acid or salt of a halogen may also be used during measurement instead of other solutions. According to the invention, any acid of the halogen group or any salt thereof is suitable, however, radioactive halogens not being desirable in practice. Consequently, the acids or salts of the halogens of fluorine, chlorine, bromine and iodine are particularly preferred, and chloride, in particular metal chloride is most preferably used. Particularly preferred, the acids or salts used according to the invention are alkali metal or alkaline earth metal salts, in particular sodium, potassium or lithium salts.

According to a particularly preferred embodiment of the present invention, the aqueous composition comprises at least one acid or salt selected from the group consisting of HCl, HF, HBr, HJ, NaCl, NaF, NaBr, NaJ, KCl, KF, KBr, and KJ. The aqueous composition comprising at least one acid of a halogen or its salt may, in addition to the at least one acid or its salt, comprise further materials such as other acids or salts. Particularly preferred are materials having a buffering function (e.g. disodium hydrogen phosphate, potassium dihydrogen phosphate, carbonate).

According to a further preferred embodiment of the present invention, the solid carrier is treated with the aqueous composition for at least 1, preferably for at least 2, more preferably for at least 5, more preferably for at least 10, more preferably for at least 20 minutes. According to the invention it has been shown that the fluorescence-enhancing effect of the carrier coated with at least one metal is particularly strong when the solid carrier is incubated for at least 1 minute with the aqueous composition comprising at least one acid of a halogen or its salt, preferably at room temperature (22° C.). If incubation is conducted at higher temperatures (e.g. between 30° C. and 40° C.), incubation time can be correspondingly reduced (e.g. at least 30 seconds). If incubation, on the other, takes place at lower temperatures (e.g. between 10° C. and 20° C.), incubation time may be correspondingly extended (e.g. at least 2 minutes).

According to a further preferred embodiment of the present invention, the metal layer on the solid carrier has a thickness of 10 nm to 200 nm, preferably of 15 nm to 100 nm. Particularly preferred, the thickness of the metal layer on the solid carrier is 10 nm to 190 nm, preferably 10 nm to 180 nm, preferably 10 nm to 170 nm, preferably 10 nm to 160 nm, preferably 10 nm to 150 nm, preferably 10 nm to 140 nm, preferably 10 nm to 130 nm, preferably 10 nm to 120 nm, preferably 10 nm to 110 nm, preferably 10 nm to 100 nm, preferably 10 nm to 90 nm, preferably 10 nm to 80 nm, preferably 10 nm to 70 nm, preferably 10 nm to 60 nm, preferably 10 nm to 50 nm, preferably 15 nm to 200 nm, preferably 15 nm to 190 nm, preferably 15 nm to 180 nm, preferably 15 nm to 170 nm, preferably 15 nm to 160 nm, preferably 15 nm to 150 nm, preferably 15 nm to 140 nm, preferably 15 nm to 130 nm, preferably 15 nm to 120 nm, preferably 15 nm to 110 nm, preferably 15 nm to 90 nm, preferably 15 nm to 80 nm, preferably 15 nm to 70 nm, preferably 15 nm to 60 nm, preferably 15 nm to 50 nm, preferably 20 nm to 200 nm, preferably 20 nm to 190 nm, preferably 20 nm to 180 nm, preferably 20 nm to 170 nm, preferably 20 nm to 160 nm, preferably 20 nm to 150 nm, preferably 20 nm to 140 nm, preferably 20 nm to 130 nm, preferably 20 nm to 120 nm, preferably 20 nm to 110 nm, preferably 20 nm to 100 nm, preferably 20 nm to 90 nm, preferably 20 nm to 80 nm, preferably 20 nm to 70 nm, preferably 20 nm to 60 nm, preferably 20 nm to 50 nm.

According to the invention, the “solid carrier” may consist of any polymeric material as long as it is coatable with a metal and as long as depressions can be generated. For example, the solid polymeric carrier comprises or consists of synthetic polymers such as polystyrene, polyvinylchloride or polycarbonate, cyloolefin, polymethyl methacrylate, polylactate or combinations thereof. Basically, non-polymeric carriers such as metals, ceramics or glass would also be suitable, as long as they are coatable with a metal and as long as depressions can be generated.

The solid carrier preferably comprises at least one material selected from the group consisting of thermoplastic polymers and polycondensates.

According to a preferred embodiment of the present invention, the thermoplastic polymer is selected from the group consisting of polyolefins, vinyl polymers, styrene polymers, polyacrylates, polyvinylcarbazole, polyacetal and fluoropolymers.

The polycondensate is preferably selected from the group consisting of thermoplastic polycondensates, thermosetting polycondensates and polyadducts.

According to a particularly preferred embodiment of the present invention, the material of the polymeric solid carrier comprises organic and/or inorganic additives and/or fillers, these preferably being selected from the group consisting of TiO₂, glass, carbon, pigments, lipids, and waxes.

According to a preferred embodiment, the inventive substrate is a part of a capillary, a microtiter plate, a microfluidic chip, a test strip (for “lateral flow assays”), a slide (e.g. object slide) for fluorescence microscopy, in particular for high-resolution methods such as confocal laser microscopy according to the point-scanner principle as well as 4Pi microscopy and STED (stimulated emission depletion) microscopy, a sensor array or any other optical detector field.

Particularly preferred is the use of the inventive substrate in microtiter plates, wherein the microtiter plates may comprise 6, 12, 24, 48, 96, 384 or 1536 wells. Microtiter plates are used for various measurements and assays, which often also comprise measuring the fluorescence of samples. By providing the inventive substrate in the wells of microtiter plates, the fluorescence yield of the samples may be increased significantly. The substrates can be introduced into and fixed in the wells by means of various methods. Here, the substrates are preferably fixed in the wells by means of adhesion, welding techniques (e.g. laser welding), and thermal bonding.

According to a particularly preferred embodiment of the present invention, the solid carrier comprises or consists of a cyclo-olefin copolymer or cyclo-olefin polymer and is part of a microtiter plate or part of the wells of a microtiter plate. It has been shown that COP 1060R (Zeonor° 1060R) is particularly well suited. Here, the carrier is preferably coated with 10 to 60 nm, preferably up to 40 nm, of a metal (e.g. silver).

Certain measurements with fluorescent substances such as fluorophores are conducted in capillaries. It is thus preferred to provide the inventive substrates in capillaries. One exemplary application is cytometry or flow cytometry, where the number or also the type of fluorescent cells or fluorescently labeled cells are determined by means of fluorescence measurement.

Numerous fluorescence measurement applications are conducted in microfluidic chips (e.g. as “lab-on-a-chip” application), where the inventive substrates may be provided in the detection area of such chips. The inventive substrates may also be provided in conventional cuvettes. This also allows a significant increase of the fluorescence yield in fluorescence measurements, so that very small amounts of fluorescent materials in a sample can be measured. According to the invention, any cuvette shape may be used. The inventive substrates may also be used in the detection area (“detection line”) of test strip systems (“lateral flow assays”) that may be used for rapid tests or in-field tests (point of care), in order to enhance the fluorescence of a labeled analyte (e.g. a fluorescently labeled antibody) and thus increase the sensitivity of the test.

According to a preferred embodiment of the present invention, the luminophore is produced by an enzyme.

Luminophores are chemical compounds that react to generate a product in case of certain reactions (e.g. oxidation with oxygen or hydrogen peroxide). During such a reaction, electromagnetic radiation in the ultraviolet and/or visible range is emitted. According to the invention, luminophores may be produced with the aid of one or more enzymes. Here, these enzymes are able to react precursors of luminophores in order to convert these precursors into energy-rich and instable luminophores.

Preferably the enzyme(s) used according to the invention is/are selected from the group of peroxidases and oxygenases, preferably from the group consisting of horseradish peroxidase (HRP), alkaline phosphatase (ALP) and luciferase. These enzymes are able to react certain compounds in a way so that they emit electromagnetic radiation. Suitable chemiluminescent compounds are well-known to the skilled person (see e.g. “Chemiluminescence in Organic Chemistry”, Karl-Dietrich Gundermann Frank McCapra, Springer-Verlag Berlin Heidelberg 1987,ISBN 978-3-642-71647-8) and are selected according to the enzyme.

According to a further preferred embodiment of the present invention, the luminophore is selected from the group consisting of luminol and its derivatives, 1,2-dioxetane, acridinium esters and luciferins. In addition to one or more of these luminophores, oxalic acid derivatives, preferably oxalic acid arylester, such as bis(2,4,6-trichlorphenyl)oxalate (TCPO), bis(2,3-dinitrophenyl)oxalate (DNPO) or bis(2,4,5-trichlorophenyl-6-carbopentoxyphenyl) oxalate (CPPO), may be used, from which is known that they may, when reacted with peroxides, e.g. hydrogen peroxide, form instable peroxyoxalates, e.g. 1,3-dioxetandione, which may emit e.g. UV light during their decomposition reaction.

The inventive enzyme(s) producing luminophores is/are preferably directly and/or indirectly bound to the substrate. Therefore the inventive substrate may, for example, be coated with enzymes that are able to produce luminophores. This is particularly advantageous when determining analytes on a surface (e.g. enzyme-linked immunosorbent assay (ELISA)).

Depending on the field of application, the enzyme may be bound to the substrate directly or indirectly via one or more further molecules. Methods for binding such molecules to metal structures are well known. In the simplest case, binding is achieved through physical chemical adsorption (mediated via ionic and hydrophobic interactions) of the proteins to the metal surface (e.g. Nakanishi K. et al. J Biosci Bioengin 91(2001):233-244). Covalent methods for immobilizing proteins after derivatization of the metal surfaces are also known (e.g. GB Sigal et al. Anal Chem 68(1996):490-7).

According to a preferred embodiment of the present invention, the enzyme is bound to the substrate indirectly via a carrier molecule selected from the group consisting of antibodies, antibody fragments, preferably Fab, F(ab)₂ or scFv fragments, nucleic acids, lipids, virus particles, aptamers and combinations thereof.

A further aspect of the present invention relates to a method for enhancing the chemiluminescence of one or more luminophores produced in a chemiluminescence reaction in an aqueous solution comprising the step of contacting the aqueous solution with a substrate as defined above.

In the inventive method, the light released by a luminophore in an aqueous solution may be enhanced by contacting this solution with the inventive substrate. This increases the sensitivity of reactions, e.g. by producing a luminophore. As an example, the detection of blood residues by means of luminol may be mentioned. Here, blood may be detected in aqueous samples or in samples rinsed or dissolved in an aqueous solution that contain only very small amounts of blood.

A further aspect of the present invention relates to a method for determining or quantifying at least one analyte in an aqueous sample, comprising the following steps:
a) contacting the sample with an inventive substrate comprising an analyte-binding molecule directly or indirectly bound to the substrate,
adding at least one additional analyte-binding molecule to which at least one enzyme is directly or indirectly bound that produces one or more luminophores from one or more substrates in a chemiluminescence reaction, and
c) measuring the light emission resulting from the chemiluminescence reaction in step b).

The inventive method for determining or quantifying at least one analyte in an aqueous sample is based on detection methods that are well known to the skilled person (e.g. ELISA), with the difference that the aqueous sample with the analyte to be determined and/or quantified is contacted with an inventive substrate also disclosed in WO 2017/046320. The presence of this substrate allows a significant enhancement of the light released in the course of a chemiluminescence reaction.

In a preferred inventive method, an aqueous sample is contacted with an analyte-binding molecule (e.g. antibody or antibody fragment) that is bound to the substrate by means of a known method. Thus, the analyte molecules present in the aqueous sample are bound indirectly to the substrate surface. In the course of a washing step, the analytes not indirectly bound to the substrate surface are removed. By means of a further analyte-binding molecule, at least one enzyme is also bound indirectly to the inventive substrate. After a further washing step, in which the further analyte-binding molecule not bound to an analyte is removed, a substrate is added from which the enzyme can produce a luminophore that releases light in one of the final steps of the method. This light can be determined qualitatively or quantitatively with conventional methods.

The luminophore is—as already mentioned at the beginning—produced by an enzyme in a chemiluminescence reaction. According to a preferred embodiment of the present invention, the enzyme is selected from the group consisting of horseradish peroxidase, alkaline phosphatase, luciferase and generally hydrolytic enzymes.

According to a further preferred embodiment of the present invention, the luminophore is selected from the group consisting of luminol and its derivatives, 1,2-dioxetane, acridinium esters or luciferins.

Preferred combinations of enzymes and substrates may be selected from the group consisting of horseradish peroxidase/luminol, alkaline phosphatase/1,2-dioxetanes and luciferase/luciferin.

According to a preferred embodiment of the present invention, the substrate-bound and at least one further analyte-binding molecule is selected from the group consisting of antibodies, antibody fragments, preferably Fab, F(ab)′2 or scFv fragments, nucleic acids, and combinations thereof.

According to a further preferred embodiment of the present invention, the enzyme is bound indirectly via a carrier molecule selected from the group consisting of antibodies and antibody fragments, preferably Fab, F (ab) ′2 or scFv fragments.

According to a preferred embodiment of the present invention the light emission in step b) is measured at a wavelength of 280 nm to 850 nm.

According to a particularly preferred embodiment of the present invention, the light emission in step b) is measured at a distance of more than 40 nm, preferably more than 50 nm, from the substrate.

The present invention will be explained in further details with reference to the following figures, without, however, being limited to these.

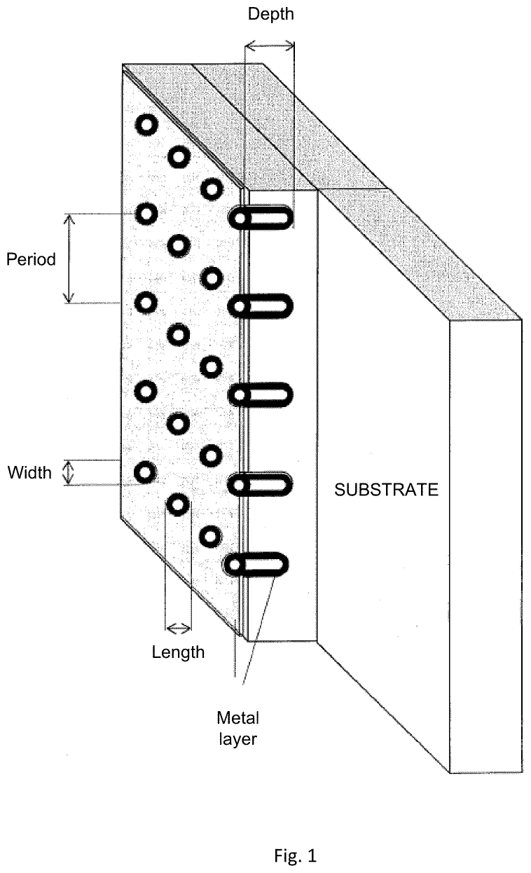

FIG. 1 shows the inventive substrate comprising a solid carrier that is coated with a metal layer. The solid carrier has depressions having a depth, a width and a length. The depressions are positioned on the solid carrier at a certain distance (period) from each other.

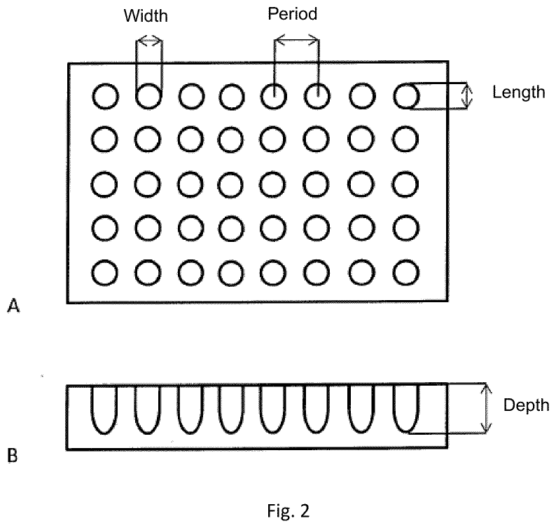

FIG. 2 shows a plan view (A) and a cross section (B) of an inventive solid carrier. The depressions on the solid carrier are characterized by a width, a length and a depth and are at a certain distance (period) from each other.

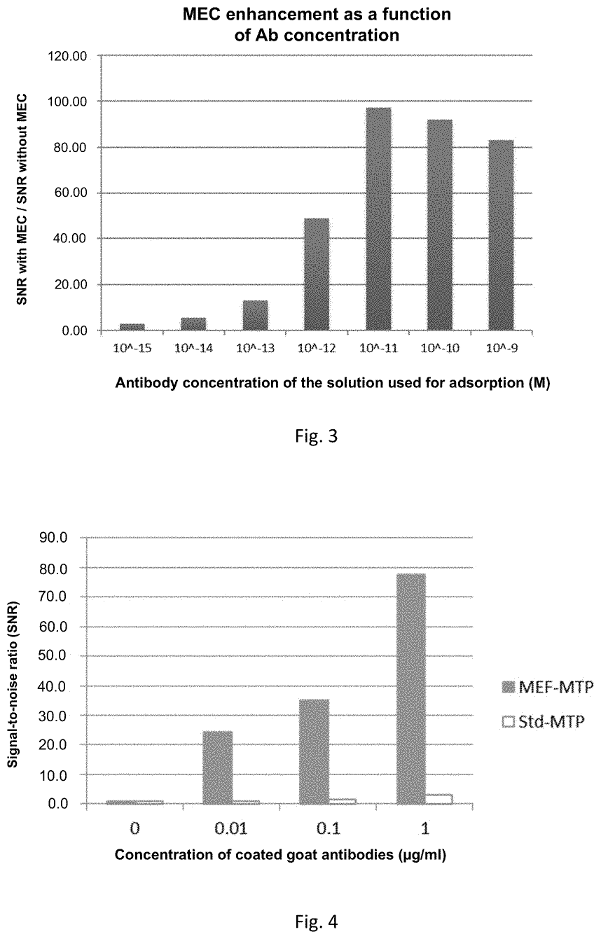

FIG. 3 shows the MEC enhancement as a function of the antibody concentration.

FIG. 4 shows the signal-to-noise ratios in a 2-step assay setup on MEF and standard MTPs. Table 2 and the following graphics show the achieved SNRs or MEC enhancements as a function of the concentration of the coated goat antibody.

### EXAMPLES

By means of the examples described below it was examined whether the substrates developed for metal-enhanced fluorescence are also suitable for improving the signal-to-noise ratio of chemiluminescence measurements.

For the following examples, microtiter plates having the structure disclosed in AT 517 746 at their bottom were used. Particularly, silver-coated polymeric carriers with a plurality of depressions separated from one another and having a diameter of 0.4 μm, a period (i.e. distance between two depressions) of 1 μm, and a depth of 0.7 μm were applied to the bottom of the microtiter plates. Commercially available microtiter plates of the company Greiner (Austria) were used for comparison purposes in order to determine the extent of the enhancement effect achieved.

### Example 1:Direct Detection of Adsorbed, Enzyme-Labeled Antibodies

The simplest method for detecting an MEC (“metal-enhanced chemiluminescence”) enhancement effect adsorbing an enzyme-labeled antibody to the bottom of a microtiter plate described above and, after a washing step, detecting the bound antibody by means of a chemiluminescence substrate for the respective enzyme.

Procedure
50 μl of a donkey-anti-goat-antibody (Sigma, SAB3700287, 1 mg/ml) dilution in 50 mM phosphate buffer/100 mM NaCl with concentrations of 10⁻⁹ to 10⁻¹⁵ mol/L were incubated for 2 h at RT in the dark on the MEF or Greiner 1×8 HB strip MTPs (VWR, 737-0195).
The content of the wells (depressions on microtiter plates) was discarded and the plates were washed 3×with 200 μl 50 mM phosphate buffer/100 mM NaCl/0.1% TritonX100.
According to the manufacturer specifications (BM Chemiluminescence ELISA Substrate Kit, Sigma, 11759779001), 10 μl of the chemiluminescence substrate were mixed with 100 μl of the enhancer and 890 μl of the assay buffer, both included in the kit. The substrate contained in the kit is CSPD (disodium 3-(4-methoxyspiro{1,2-dioxetan-3,2′-(5′-chloro)tricyclo[3.3.1.1^{3,7}]decan}-4-yl)phenyl phosphate), which is converted into an instable dioxetane by the ALP and has its emission maximum at 477 nm. The enhancer used for enhancing the quantum yield in this kit was Emerald II.
150 μl of this reaction mixture were pipetted onto the MTPs and the emerging chemiluminescence signal was monitored with a SPARK microtiter plate reader by TECAN.

Results

Temporal Evolvement of the Signal-To-Noise Ratio (SNR, Chemiluminescence of a Well with Antibodies/Chemiluminescence without Antibodies)

As may be seen in Table 1, the SNR only increases over time in the microtiter plates with a structured bottom as described at the beginning (MEF-MTP), while it stagnates or even decreases on a standard microtiter plate (“Greiner MTP”) by Greiner.

TABLE 1
SNR over time
Signal-to-noise ratio-MEC enhancement

| | t = 30 sec | | | t = 300 sec | | |
|---|---|---|---|---|---|---|
| Antibody | MEF- | Greiner | | MEF- | Greiner | |
| (M) | MTP | MTP | MEC | MTP | MTP | MEC |
| 0 | 1.0 | 1.0 | 1.0 | 1.0 | 1.0 | 1.0 |
| 10\hat{ }-15 | 1.9 | 1.0 | 1.9 | 3.2 | 1.0 | 3.2 |
| 10\hat{ }-14 | 4.5 | 2.0 | 2.3 | 10.2 | 1.9 | 5.5 |
| 10\hat{ }-13 | 18.4 | 4.2 | 4.4 | 50.2 | 3.8 | 13.1 |
| 10\hat{ }-12 | 147.4 | 8.6 | 17.1 | 427.9 | 8.8 | 48.7 |
| 10\hat{ }-11 | 1385.8 | 35.3 | 39.2 | 3564.6 | 36.7 | 97.2 |
| 10\hat{ }-10 | 8144.8 | 211.5 | 38.5 | 16237.9 | 176.3 | 92.1 |
| 10\hat{ }-9 | 16176.0 | 441.0 | 36.7 | 25781.6 | 309.8 | 83.2 |

This may be explained by the fact that due to the MEC, which increases the quantum yield, quenching of the bulk solution enriching itself with luminophores is prevented. Consequently, the detection of the adsorbed antibody on the MEF-MTP becomes more and more sensitive over time compared to a standard MTP and allows a reduction of the detection limit by at least a power of ten.

Dependence on Concentration and Extent of MEC

The simplest way to conduct a quantification of the enhancement extent is by comparing the SNRs on MEF and standard MTPs.

This showed a clear dependence of the extent of MECs on the concentration (data after 300 sec measurement time), as shown in FIG. 3. This enhancement increased up to a concentration of 10⁻¹¹ of molar antibody adsorption solutions to almost the 100-fold, but then starts decreasing again, which is probably also due to increasing quenching. Even with low concentrations in the sub-picomolar range, there is still observable a clear 3- to 13-fold enhancement of the SNR of a standard MTP.

### Example 2: Indirect Detection of Adsorbed, Non-Labeled Antibodies

The extent of the MEF (“metal-enhanced fluorescence”) effect strongly depends, inter alia, on the distance of the fluorophores from the nanostructure (s. also Hawa et al. Analytical Biochemistry 549(2018): 39-44).

In the case of an enzyme-catalyzed MEC test, contrary to MEF tests with e.g. directly fluorescently labeled antibodies, the produced luminophore diffuses away from the surface and may thus only be enhanced as long as it is on the surface. In order to show that a luminescence enhancement is also possible in an assay setup over two protein layers (at least approx. 10-15 nm), the experiment described in Example 1 was modified in that first, a goat antibody was applied to the MEF-MTP, which was then detected after a blocking step with an ALP-labeled anti-goat antibody.

Procedure:
50 μl of a goat-antibody (Jackson, 111-005-008, 2 mg/ml) dilution in 50 mM phosphate buffer/100 mM NaCl with concentrations of 0-1 μg/ml were incubated over night at 4° C. in the dark on the MEF or Greiner 1×8 HB strip MTPs (VWR, 737-0195).
The content of the wells was discarded and the plates were washed 3× with 200 μl 50 mM phosphate buffer/100 mM NaCl/0.1% TritonX100.
Blocking of unspecific binding was conducted by incubation for 2 hours at RT with 100 μl of 50 mM phosphate buffer/100 mM NaCl/0.1% TritonX100 with 5% polyvinylpyrrolidone (5% PBSPTx)
After a further washing step, the plates were incubated with 50 μl of a 30 ng/ml solution of the ALP-labeled anti-goat antibody also used in item 2 for 2 h at room temperature.
After a final washing step, again 150 μl of the luminophore reaction mixture described in Example 1 were added and the emerging chemiluminescence signal was monitored with a SPARK microtiter plate reader by TECAN.

Results

A quantification of the extent of enhancement was again conducted by comparing the SNRs on MEF and standard MTPs (Greiner MTP). Table 2 and FIG. 4 show the obtained SNR values and MEC enhancements as a function of the concentration of the coated goat antibody.

TABLE 2
Signal-to-noise ratio in 2-step assay setup
Signal-to-noise ratio

| Antibody | | | |
|---|---|---|---|
| (μg/ml) | MEF-MTP | Std-MTP | MEC |
| 0 | 1.0 | 1.0 | n.a. |
| 0.01 | 24.4 | 1.0 | 24.4 |
| 0.1 | 35.2 | 1.6 | 22.0 |
| 1 | 77.5 | 2.9 | 26.7 |

The enhancement effect is even observable over two protein layers. This is surprising because compared to the MEF with surface-bound fluorophores, the enzyme-produced luminophores diffuse and move further away from the surface. In any case, sensitivity increases by a factor of 20-30 are to be considered analytically relevant since they seem to increase with increasing coating concentrations.

Discussion

The structures developed for metal-enhanced fluorescence are thus also suitable for metal-enhanced chemiluminescence.

This is very surprising because the reach of the effect is only 40-50 nm (distance from the surface) and the chemiluminescent substrate produced by the enzyme diffuses away from the surface. Apparently enhancement is so strong that averaged over time, enough molecules are present close to the surface. Also the observed extent of the MEC is unexpected and has not been described in the literature so far.

1. A method for enhancing the chemiluminescence of one or more luminophores produced in a chemiluminescence reaction, comprising:
providing a substrate that comprises a solid polymeric carrier with a plurality of depressions separated from one another, wherein the solid polymeric carrier is at least partially coated with at least one metal.

2. The method according to claim 1, wherein the depressions in the plurality of depressions have a distance from each other of 0.2 μm to 2.5 μm.

3. The method according to claim 1, characterized wherein the depressions in the plurality of depressions have a length and a width, and wherein the ratio of the length to the width ranges from 2:1 to 1:2.

4. The method according to claim 1, wherein the depressions in the plurality of depressions have a length and a width, wherein the length is 0.1 μm to 2 μm and the width is 0.1 μm to 2 μm.

5. The method according to claim 1, wherein the depressions in the plurality of depressions have an essentially round shape.

6. The method according to claim 1, wherein the depressions in the plurality of depressions have a depth of 0.1 μm to 5 μm.

7. The method according to claim 1, wherein the at least one metal comprises at one or more than one at least partial metal layers arranged above one another; and optionally the at least one metal has a thickness of 10 nm to 200 nm.

8. The method according to claim 1, wherein the at least one metal is chosen from silver, gold, aluminum, chrome, indium, copper, nickel, palladium, platinum, zinc, tin, and alloys comprising one or more thereof.

9. The method according to claim 1, wherein the solid polymeric carrier comprises at least one material chosen from thermoplastic polymers, polycondensates, polyolefins, vinyl polymers, styrene polymers, polyacrylates, polyvinylcarbazole, polyacetal polymers, fluoropolymers, thermoplastic polycondensates, thermosetting polycondensates, and polyadducts, and optionally comprises one or more of TiO₂, glass, carbon, pigments, lipids, and waxes.

10. The method according to claim 1, wherein the one or more luminophores are produced by an enzyme.

11. The method according to claim 10, wherein the enzyme is chosen from horseradish peroxidase, alkaline phosphatase, luciferase and hydrolytic enzymes.

12. The method according to claim 1, wherein the one or more luminophores are chosen from luminol and its derivatives, 1,2-dioxetane, acridinium esters and luciferins.

13. The method according to claim 10, wherein the enzyme is directly and/or indirectly bound to the substrate.

14. (canceled)

15. A method for determining or quantifying at least one analyte in an aqueous sample, comprising:
a) contacting the aqueous sample with a substrate that comprises a solid polymeric carrier with a plurality of depressions separated from one another, wherein the solid polymeric carrier is at least partially coated with at least one metal; the substrate further comprising an analyte-binding molecule directly or indirectly bound to the substrate,
b) adding at least one additional analyte-binding molecule to which at least one enzyme is directly or indirectly bound that produces one or more luminophores from one or more precursors in a chemiluminescence reaction, and
c) measuring the light emission resulting from the chemiluminescence reaction.

16. The method according to claim 15, wherein the at least one enzyme is chosen from horseradish peroxidase, alkaline phosphatase and luciferase.

17. The method according to claim 15, wherein the one or more luminophores are chosen from luminol and its derivatives, 1,2-dioxetane, acridinium esters and luciferins.

18. The method according to claim 15, wherein the analyte-binding molecule and the at least one additional analyte-binding molecule are chosen from antibodies, antibody fragments, Fab, F(ab)′2 fragments, scFv fragments, nucleic acids, aptamers, and combinations thereof.

19. The method according to claim 15, wherein the at least one enzyme is indirectly bound via a carrier molecule chosen from antibodies and antibody fragments, Fab, F(ab)′2 fragments, scFv fragments, nucleic acids, aptamers, and combinations thereof.

20. The method according to claim 15, wherein the light emission is measured at a wavelength of 280 nm to 850 nm.
